(19) United States
(12) Patent Application Publication
(10) Pub. No.: US 2005/0089494 A1
(43) Pub. Date: Apr. 28, 2005

(54) Mending hair damage with polyelectrolyte complexes

(75) Inventor: Raymond Rigoletto JR., Denville, NJ (US)

Correspondence Address: Attn: William J. Davis, Esq.;Legal Department, Building No. 8, INTERNATIONAL SPECIALTY PRODUCTS, 1361 Alps Road, Wayne, NJ, 07470, US

(73) Assignee: ISP INVESTMENTS INC.

(21) Appl. No.: 10/890,651

(22) Filed: Jul. 14, 2004

Related U.S. Application Data
(60) Provisional application No. 60/514,953, filed on Oct. 28, 2003.

(51) Int. Cl. A61K 7/06; A61K 7/11

(52) U.S. Cl. 424/70.15; 424/70.16

(57) ABSTRACT

Polyelectrolyte complexes between anionic and cationic polymers are used to mend damaged hair fibers, especially damage exhibited by split ends. An improved test method designed for the assessment of the degree of split end repair is described which consists of tagging hair fibers and observing the repair process by stereomicroscopy.

### CROSS REFERENCE TO RELATED APPLICATION

This application corresponds to U.S. Provisional Patent Application Ser. No. 60/514,953, filed on Oct. 28, 2003, the complete disclosure of which is hereby incorporated by reference.

### BACKGROUND OF THE INVENTION

1. Field of the Invention

This invention relates to hair care compositions, and, more particularly, to hair repair compositions for mending damaged fibers such as split ends of hair fibers.

2. Description of the Prior Art

The technical term for split ends is trichoptilosis. It is defined as a longitudinal splitting of the hair fiber which develops after the protective cuticle has been stripped away from the end of the hair fibers as a result of either physical or chemical traumatizing of the hair. Formation of split ends develops because of the fine structure of hair and the forces that are at work in its components.

There is a sequence of mechanical events that occur in the formation of split ends. During the combing process, fibers tend to snarl as the comb approaches the tip ends of the hair. It can be observed in these entanglements that the fibers are bent to various degrees. Since hair is elliptical and has a major and a minor axis, this bend occurs parallel to the major axis. The bending effect subjects the hair to longitudinal shear stresses parallel to the major elliptical axis. The magnitude of these shear forces are parabolically distributed across the minor axial diameter of the elliptical section. As hairs are being bent, these shear stresses will result in fracture and will propagate along the major elliptical axis resulting in a split end. The fibers bend as they pass through the prongs of a comb.

The disulfide bond in hair is another factor in split end formation. Protein molecules in the cortex of the hair fiber are crosslinked by covalent disulfide bonds, which provides strength and flexibility. During chemical processing, or through environmental damage, these bonds tend to break which makes the hair more susceptible to mechanical damage. The result is that damaged hair is more prone to splits and cracks during the combing and brushing process.

Split ends are more prone in hair that has been damaged by weathering, chemical treatments, or mechanical damage. Prevention of split ends generally involves adding lubricity to the hair with cationic surfactants so that there is less friction during combing resulting in less snarling. Also, adding plasticizing agents, which allows the hair to bend with more facility, will reduce split ends. Since water is a plasticizing agent, it is expected that fewer split ends will be produced at a high relative humidity, particularly, with the application of humectants that allows for moisturization.

Lubricating agents that can prevent or minimize formation of split ends include cationic surfactants commonly found in crème rinses, e.g. cetrimonium chloride, stearalkonium chloride, dicetyldimonium chloride, and behentrimonium methosulfate. Cationic polymers are more common lubricating agents found in shampoos. These polymers include polyquaternium compounds such as polyquaternium-6, -7, -10, -11, and -28. Guar hydroxytrimonium chloride also is used as a lubricant. The mechanism by which such conditioner reduces damage during combing is based upon lubricity. The lubrication reduces the friction in the hair during combing and hence reduces the strength of the abrasive forces to which the hair is being subjected. This in turn reduces the number of entanglements during the combing process.

Plasticizing agents including humectants such as glycerin, propylene glycol, acetamide MEA, and sorbitol also are used for this purpose. An example of a substantive humectant containing a positive charge is Quaternium®-22 (Ceraphyl® 60). By treating the hair with conditioners that provide these properties it is possible to prevent the formation of split ends.

Prior Methods of Producing and Assessing Split End Mending and Prevention

A mechanical device has been described in the literature to generate split ends. It consists of a motor driven rotary arm to which two combs are attached. A hair swatch is clamped into place so that the ends of the hairs are struck by the rotary arm. A linear relationship was established by Cooper between the time of exposure and the number of split ends generated. Hair treatment such as shampoos, conditioners and mousses reduced the number of split ends produced, however, studies are not conducted to affect their repair. Prevention was determined by comparing the percentage of hairs split in the hair swatches that were treated with different conditioning preparations. No explanation was provided, however, as to how this percentage is derived, or whether it is based on a sample of fibers from the tress or based on all the fibers in the tress.

Kon et al in J. Cosmet. Sci. 20, 361-380 (1998) also developed a method for artificially inducing damage to hair in order to serve as a model for studying the effect of ingredients on hair damage. Two models are described, one for scale lift and one for split hair, both having their own characteristic method. The split hair model consists of soaking the hair in a reducing solution that extracts a certain quantity of cortical protein from the hair fiber. After a detailed procedure of washing, de-lipidation, drying, reducing, washing, re-oxidizing, and lyophilizing (freeze-drying), the hairs are viewed for splits with an SEM. They postulated that the driving force for the split is osmotic shock. With this technique it was found that keratin peptides and cationic hydroxyethylcellulose have a preventative effect on split formation. These test solutions are added in between the reducing and oxidizing steps of the test method. Their explanation for adding these ingredients in the middle of the process is the fact that they prevent expansion of the split hairs during the lyophilization stage. They mention that peptides of low molecular weight can do this because they are more penetrative, and cationic derivatives because of their high affinity for the cortical proteins. Kon mentions that the method by Cooper has low reproducibility.

Split End Repair and Prevention Methodology

U.S. Pat. No. 6,071,505 described a water soluble quaternary ammonium cellulosic derivative of controlled charge density. It mentions many uses of these novel compounds applicable in both hair and skin care products. One such use is that it can mend split ends. However, no substantiation was given in the patent and there is no test method revealed as to how split end mending is achieved.

A brochure by Amerchol Corporation stated that all grades of UCARE® Polymer can mend split ends. The method described is as follows. “ . . . individual strands of virgin brown hair were mounted on a plastic stand for each shampoo to be tested. Each hair was then shampooed by inserting the individual hair into an eyedropper containing the test formulation. The strand was then rinsed with distilled water from a squeeze bottle and the process was repeated to simulate normal shampoo procedure. The hairs were examined immediately by hand lens for mended splits, and again after drying one hour at 54° C. (129° F.). To simulate combing, each hair was “tickled” ten times with a spatula and the number of splits still mended was recorded. After one hour at room temperature, the hairs were again examined.” Results indicate that at every stage of the experiment, UCARE® Polymer containing shampoos provide for 100% mending of split ends. The control shampoo containing just TEA Lauryl Sulfate and Cocamide DEA provided for 70% mending. With this method the combing simulation did not subject the hair to enough stress that actual combing provides. Also, the 70% mending rate for the control shampoo is too high suggesting that the tickling does not provide enough stress on the fiber that actual combing does to reopen the split end.

U.S. Pat. No. 6,258,348 described a hair conditioning composition specifically for mending split ends. The composition consists of a ternary mixture of guar (cationic or amphoteric) a betaine based polyurethane surfactant, and a silicone polyurethane. This ternary blend can be incorporated into a cream rinse or a 2-in-1 shampoo to effectively mend split ends. The test method used to support mending for the example formulations in the patent consists of selecting ten split end fibers from a tress and then attaching them to a single tab tress. The single tab tress with the split end fibers are then subjected to the treatment such as shampooing and left to air dry. Split end repair is then evaluated under a magnifying glass. Percent mending is then reported. Their findings indicate that a high percentage of mending occurs with 2-in-1 shampoos containing a ternary blend of ingredients compared to the same composition with only two of the three components. One drawback with this method is that the permanence of the mend was not studied, only the initial mending. Also, combing ten fibers is not a realistic amount of hair that would impart enough stress during the combing process.

U.S. Pat. No. 4,900,545 described that a composition of panthenol, glucose, PVP and phytantriol could regenerate hairs that have been split. This result was tested on 100 hairs all of which had been split by mechanical or electrostatic pretreatment. The hairs were treated 10 minutes with the undiluted preparations of examples 1 through 7 therein. The hairs were then rinsed with tap water, dried and combed. The visually discernable split ends remaining were then determined by counting. The percentage of split ends remaining for the 7 formulas ranged from 10 to 50%. This method suffers because 100 fibers is not a sufficient mass of fibers when passed through the comb to subject the hair to sufficient bending to cause the hairs to split.

U.S. Pat. No. 6,251,379 disclosed that hair compositions containing a combination of quaternized keratose and a defined silicone derivative can mend split ends. The method to substantiate this effect consisted of using bundles of Asian type hair, treating them with the composition, and drying with combing and blow drying. The hair is then brushed another ten times. Split end mending is assessed both before and after treatment. Although there is a standard for judging the degree of the prevention of re-splitting, it appears that the assessment is qualitative in nature. One positive with this method, however, is the fact that it assesses split end repair after the hair is brushed.

U.S. Pat. No. 6,139,851 described that a hair cream based on a lower alcohol in oil type emulsion containing silicone derivatives to mend split ends. The method is the same as is described in U.S. Pat. No. 6,251,379. No quantitative assessment of split end repair was described suggesting to a greater extent than the previous patent that a qualitative assessment of split end repair was used.

Ramachandran in WO 96/32920 described an improved hair rinse composition. They claim that their hair rinse composition not only conditions hair to provide such properties as wet detangling, but also lends fixative properties and repairs split ends. The three main ingredients that comprise the rinse are quaternary ammonium salts, water insoluble acrylic or acrylate polymers and a solvent that comprises a long chain alcohol and/or alcohol ethoxylate. The solvent was used to compatibilize the quat and polymer. It is theorized, but no proof is shown, that the quaternary surfactant and polymer form a complex with each other in the solvent. It was also theorized that this complex is deposited during the rinse cycle of this hair conditioning composition which provides the function of both conditioning and styling that normally are contradictory to one another when formulated in the same product. Proof of this is supplied by running panel tests on hair swatches for ease of combing and stiffness in hair characteristic panel studies using tresses. It was found in all cases that high scores are obtained for these two hair characteristics when hair was treated with rinses containing the quat-polymer-solvent complex.

Split end mending also was measured therein using a Salon panel study. The procedure consisted in essence of a cycle of washing, drying, counting, treating, combing and counting. Counting split ends consisted of bundling the hair on the head in discrete sections. Using magnifying binoculars, 100 hairs were counted in each section and the number of split ends calculated for the whole head. Experimental results proved that the rinse containing the complex mended split ends. It was also determined that the degree of split end mending was dose related and increased with the number of treatments. They theorized that split end mending is achieved by the adhesiveness of the polymer. The thin layer of polymer that remains on the inner surfaces of the split ends after drying holds the splits together. The method relies heavily on counting split end fibers and statistical analysis of the data.

### SUMMARY OF THE INVENTION

Split ends are one manifestation of hair damage, which are caused by physical, mechanical and chemical means.

Accordingly, it is an object of this invention to provide an improved methodology for the assessment of repair of split ends.

Another object herein is to provide a new methodology using stereomicroscopy, to observe mending and subassembly of the hair fiber, as well as the permanence of the mend.

Still another object is to use methodology to indicate a chemical system, particularly a polyelectrolyte complex, for repair and permanent mending of split ends. Phase diagram work shows that there is inter-polymer interaction between the anionic Gantrez® S-97 and cationic Conditioneze® NT-20. The phase diagram teaches that there are suitable ratios of the two polymers that form a complex. This complex has a high percentage of permanently mending split ends after low stress combing in a leave-on or rinse-out treatment.

What is described herein is a hair care composition for mending damaged hair fibers comprising a polyelectrolyte complex between anionic and cationic polymers.

### IN THE DRAWINGS

FIG. 1 is a Phase Diagram Leverage Plot showing a positive interaction between S-97 and NT-20.

FIG. 2 is a Leverage Plot for S-97*NT-20 interaction.

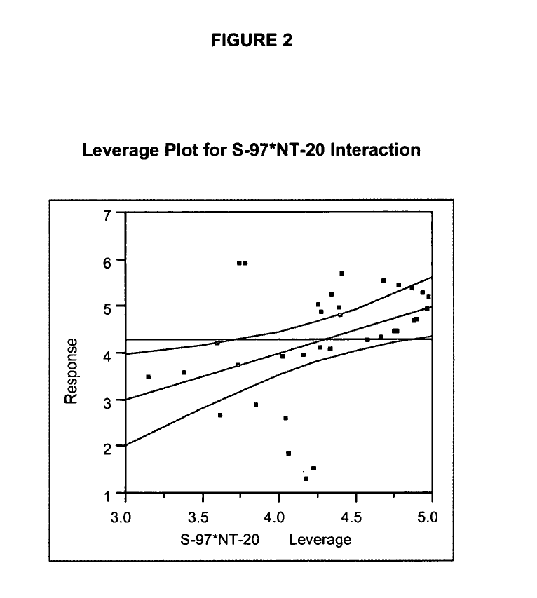

FIG. 3 is a stereomicroscope picture of a tagged fiber.

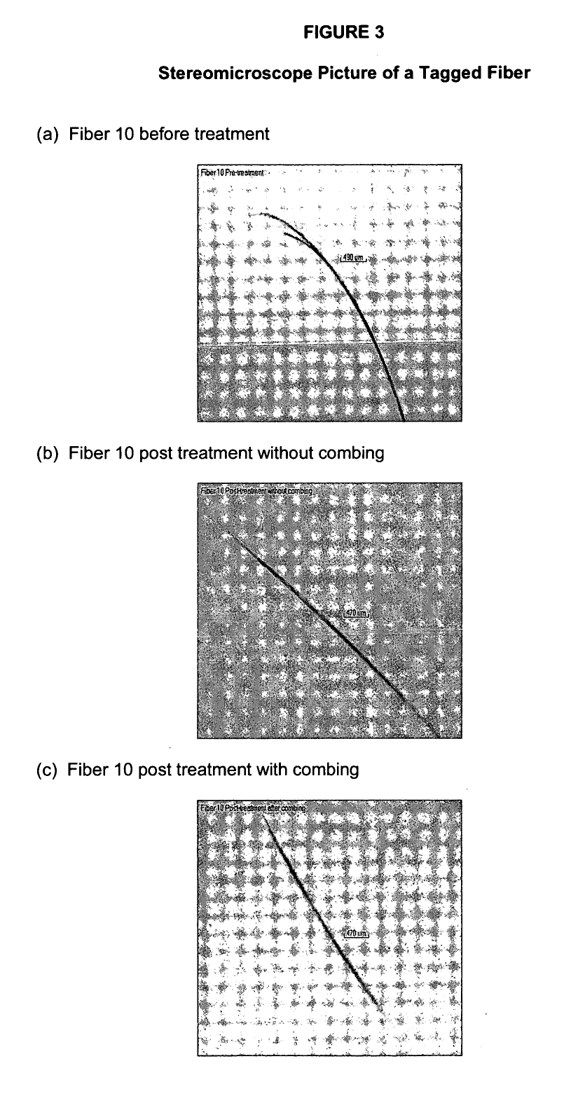

### DETAILED DESCRIPTION OF THE INVENTION

The scope of this application deals with one form of hair repair, specifically split end mending. This invention provides an improved method for assessing split end repair to substantiate the ability of a polyelectrolyte complex between Gantrez® S-97 and Conditioneze® NT-20 to repair this type of hair damage. The major feature of the improved test method used for the assessment of split end repair consists of tagging particular split ends fibers in a hair tress. The tagging allows the study of the fate of that particular split end, and importantly subjecting it to normal combing stresses in order to determine the permanence of the mend. This invention, however, is concerned with split end repair and mending rather than prevention. Two mechanisms are at work in the repair process. The first of these is film formation preferably from a substantive polymer. When the fiber is wet the split end will fuse together through surface tension forces. Also, at this stage the cationic polymer is adsorbed onto the surface of the hair between the splits as they are being brought together. When the fiber is dried the polymer coating around the shaft of the fiber as well as the polymer adhered between the splits help fuse the split end together through their adhesive qualities. Examples of cationic polymers are the same as for those mentioned for split end prevention. Anionic polymers, such as the ethyl ester of PVM/MA Copolymer, are more applicable for leave in applications since they do not have an affinity to hair in rinse off applications on their own. The other mechanism of split end repair includes binding of the protein molecules within the cortex of the fiber. The one class of ingredient cited in the literature that repairs hair in this way is hydrolyzed animal or vegetable protein. In this case its molecular weight is small enough so that it can penetrate into the cortex of the hair. Since they have various functional groups based on their amino acid content they bind to the protein in the hair. The result is an increase in the strength of the hair as measured by its tensile strength. This binding process is also at work in the split end mending process where the exposed protein of the cortex in between the split end is bound together with the hydrolyzed protein.

Structure-property relationships are important factors to consider in this process. One has to theorize how the ingredient will interact with the fine structure and chemistry of hair to determine if it is a candidate for experimentation. One theory of split end mending includes polymer adhesion in conjunction with cationic polymer crosslinking. In this case a polymer solution wets out the fiber and the split end closes through the surface tension of the water. When the polymer solution dries the split ends are held together both by the adhesion of the polymer for the hair as well as crosslinking the polypeptide chain with the cationic polymer. In order to incorporate the two properties of cationicity and fixative in one system one could envision a blend of an anionic and cationic polymer or better still a complex where synergistic effects would be evident. A complex defined here is when two polymers interact with non-covalent bonds such as ionic, hydrogen bonding, or through an associative mechanism of hydrophobic groups on the molecule. In this case the two oppositely charged polymers interact through their anionic and cationic charges to form a polyelectrolyte complex. A theory of how this complex would work in hair repair is based on the fact that the cationic polymer would be tenacious to the hair and if it is complexed to an anionic fixative polymer the cationic polymer would render the anionic substantive as well. This mechanism would be advantageous in rinse off applications such as shampoos and conditioners as well as leave on products such as lotions and gels. In leave in products the durability of the mend would also be enhanced by the lubricating properties of the cationic conditioning polymer. During the rinse cycle in a rinse out application such as a shampoo or conditioner, there would be some affinity of the anionic fixative polymer for the hair through its complexation with the cationic polymer. Many polymers of differing charge complex to impart added benefits to styling/conditioning products, however, split end repair is not described. In fact, it may not have been discovered to date whether these polymer complexes have their effect in treating hair damage and specifically split end repair.

One preferred example of an anionic/cationic polymer complex described in this invention in a leave-in or rinse-out treatment is a complex of Gantrez® S-97 and Conditioneze® NT-20. A phase diagram study suggests a suitable ratio of anionic-to-cationic polymer to form the desired complex. In this system, the Conditioneze® NT-20 will be substantive to the hair surface and bind the sub-assemblies of the split end based on it being a cationic polymer. Gantrez® S-97 which has been neutralized carries a negative charge, which, at the appropriate ratio with Conditioneze® NT-20, will form a complex. Gantrez® S-97 is the parent compound to Gantrez ES-225 which has been used traditionally as a hair spray polymer adding fixative properties to hair through its adhesiveness. Control experiments were performed to determine if the complex is doing the work of hair mending or just the additive effects of the individual polymers. This was achieved by testing the polymers individually and testing blends that do not form the complex. The complexes full potential is realized in a rinse-off situation, which would give further novelty to the idea. Suitably, other polymer combinations that form a similar complex would work to repair hair split ends by the mechanisms described above.

While not being bound by the mechanism of action herein, in a leave-in application, the product is believed to function by cationic-anionic-crosslinking, cationic lubrication for prevention, the anionic polymer for adhesion, and the cationic polymer to hold the anionic polymer on the substrate during drying.

The improvement in this method will become evident by considering those methods previously described. The qualities of this method over previous methods are that the tagging process is more precise through its assessment of split end repair, whereas previous methods are more qualitative in their assessment. Since the tagged fibers are part of the tress they are subjected to combing forces that are more realistic in assessing the permanence of the repair of the split end.

In the rinse-out mode, the cationic polymer is believed to attach to hair during the rinse cycle, which brings the anionic polymer with it as an auxiliary effect, it acts as a delivery system for any silicone component of the formulation.

This invention particularly involves the formation of a polymeric anionic and cationic complex between neutralized anionic Gantrez® S-97 polymer and cationic Conditioneze® NT-20 polymer. This polyelectrolyte complex was tested for its effectiveness as a split end mender when subjected to low combing stresses. Three controls were tested (1) a blend of Conditioneze® NT-20 and Gantrez® S-97 at 1% active each (which resulted in an un-complexed solution), (2) a 2% NT-20 solution, and (3) a 2% S-97 solution. The results of these controls indicated that effectiveness of split end mending was due to the complex formation of the polymer, rather than a solution containing both polymers, or only one polymer.

The structures of the two polymers in the polyelectrolyte complex of the invention are: 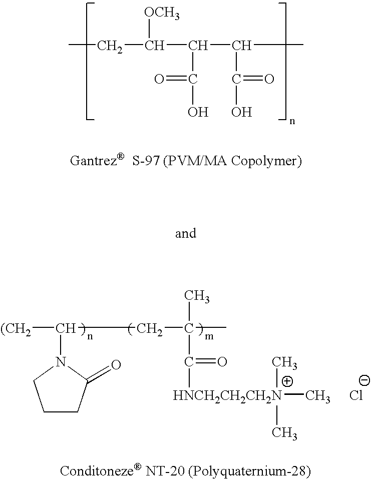
Description of Invention
Materials:
Hair tresses from International Hair Importers. Hair type is virgin brown that has been sewn to 1¾″ across with 3.5 grams of loose hair, 6.5″ in length from bottom of binding to tip.
Plastic comb from Sally's Beauty Supply, having a fine comb density of 8 prongs per centimeter.
Tweezers.
Hand held magnifying glass (1.2×).
Tags made from thin strips of Scotch tape.
Permanent Marker.

Alberto VO5 Normal Shampoo. Ingredients: WATER, SODIUM LAURYL SULFATE, COCAMIDOPROPYL BETAINE, AMMONIUM CHLORIDE, TOCOPHERYL ACETATE (VITAMIN E), PANTHENOL (PRO-VITAMIN B5), RETINYL PALMITATE (VITAMIN A), ASCORBIC ACID (VITAMIN C), OCTOXYNOL-9, ERGOCALCIFEROL (VITAMIN D), SODIUM LAURETH SULFATE, TETRASODIUM EDTA, DMDM HYDANTOIN, FRAGRANCE, CITRIC ACID, YELLOW NO. 6

Equipment

Thermal/Mechanical Styling Apparatus with two brushes
Nikon SMZ 1500 Stereomicroscope
Linksys 2.2 program for digital imagery from microscope
   Linksys for Windows
   Linkam Scientific Instruments Inc.
   8 Epsom Downs Metro Center, Waterfield
   Waterfield, Tadsworth, Surrey
   KT205HT England
      Procedures:
      Process for Creating, Treating, and Measuring Split Ends on Tress using a Polymer Complex:
      Production of Split Ends
1. Rinsed the hair for 10 seconds.
2. Combed the hair 10 times using the fine teeth to rid the tress of excess water and comb-out initial snags.
3. Used the thermal/mechanical styling apparatus for 20 minutes at 75 rpm with low fan speed, high heat from a hair dryer for a total of 3,000 (150×20) combings. (Two brushes per revolutions).
4. Point blow dryer at end of tress.
5. Make sure brush hits tress at least ¾ of tress length.
6. Repeat steps 1-5 two more times. Inspect tress for split end density to determine if steps 1-5 should be repeated. Density determined by counting number of splits per 100 fibers.
7. Total number of combings equaled ˜9,000.
   Pre-Treatment
1. Washed hair tress twice with 10% solution of Alberto VO5 Normal Shampoo and air-dried.
2. Isolated 20 split ends with hand held lens and labeled them at their root ends 1 through 20. Choose splits uniformly distributed through tress.
3. Carefully draw a dot with a visible permanent marker (red or green is preferable) slightly before the beginning of the split to later determine if the split prematurely broke off after combing.
4. Took pictures under stereomicroscope (20×). (Designated as before treatment).
   Post Treatment Without Combing
5. Added 0.5 g of formula to damp hair tress and worked it through so that it was totally distributed through hair. Let air dry under hood.
6. Gently stroked tress with fingers to break crust.
7. Inspected tagged fibers for split end repair with hand held lens and with the naked eye.
8. Took pictures of tagged fibers under stereomicroscope. In order to allow for easy, accurate comparisons, made sure hair was oriented in same direction as in initial picture. (Designated as post treatment before combing).
9. Counted tagged fibers for split ends as observed from pictures taken under 20× using stereomicroscope.
   Post Treatment with Combing
10. Combed tress twenty times with fine teeth of comb.
11. Inspected tagged fibers for split end repair with hand held lens and with the naked eye.
12. Took pictures of fibers under stereomicroscope as in step 8. (Designated as post treatment after combing).
13. Inspect tress to see permanent mark to guard against false positives.
14. Counted tagged fibers for split ends as observed from pictures taken under 20× using stereomicroscope (see example of photographs in appendix).
   Steps for Taking Pictures using Linksys:
1. Focus specimen under microscope under setting 2, which is 20-power magnification.
2. On program, click on Set Up, then Video and Measurement. Type in image description, and select capture 2. The microscope should be calibrated before this is performed.
3. Take picture (click on icon).
4. Draw scaled line by clicking on calibration icon and drawing scaled line on picture.
5. Save image to file by clicking on File, then Save As, then Image.
   Formation of the Complex

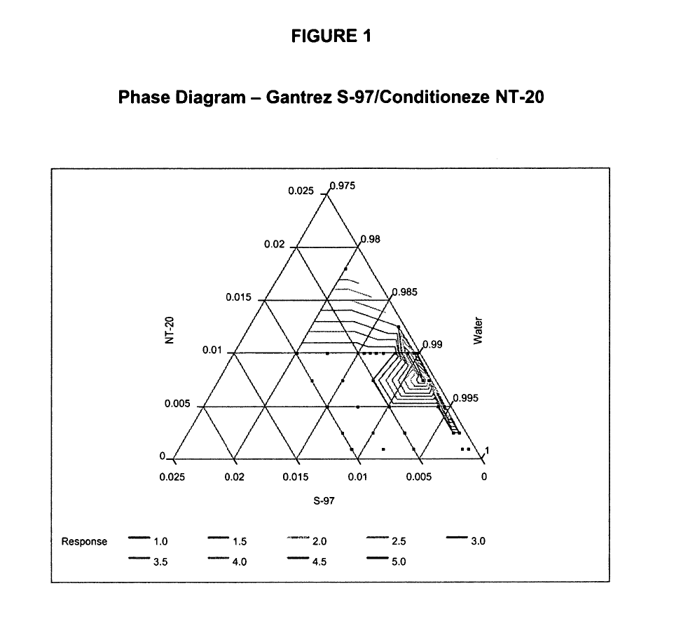

In order to determine the ratio of S-97 and NT-20 where complexation occurs a phase diagram of the two ingredients was constructed (FIG. 1) of compositions of the following stock solutions:

INGREDIENTS

| Batch A: 2% active S-97 Solution | % W/W | SUPPLIER |
|---|---|---|
| Water | 93.0 | |
| Sodium Hydroxide (10.00% active) | 5.0 (0.50% active) | |
| Lot #: B0110035 | | |
| PVM/MA Copolymer | 2.0 (2% active) | ISP |
| (Gantrez ® S-97) (100% active) | | |
| | 100.00% | |

Note:
In order to neutralize the Gantrez S-97, a 1:2.7 ratio of polymer to 10% NaOH solution was used to produce a pH of 7. A ratio of 1:2.5 of polymer (S-97) to 10% NaOH solution produced a pH of 6.68.

Procedure:
1. Add water to main tank. Mix with propeller blade.
2. Sprinkle Gantrez S-97 into vortex.

3. Add Sodium Hydroxide and mix until uniform.

Physical Properties:

| | |
|---|---|
| Appearance: | hazy serum |
| pH: | 6.68 |
| Viscosity: | 177.1 (16.6%) (Brookfield DVII + RVT, |
| | LV-1, 60 rpm, ambient) |

| Batch B: 2% active NT-20 Solution | % W/W | SUPPLIER |
|---|---|---|
| Water | 90.0 | |
| Polyquaternium-28 | 10.0 (2% active) | ISP |
| (Conditioneze ® NT-20) (20% active) | | |
| | 100.00% | |

Procedure:
1. Add water to main tank. Mix with propeller blade.

2. Add Conditioneze NT-20. Mix until uniform.

Physical Properties:

| | |
|---|---|
| Appearance: | clear liquid |
| pH: | 7.22 |
| Viscosity: | 95.8 (45.9%) (Brookfield DVII + RVT, |
| | UL, 00, 30 rpm, ambient) |

Procedure for Making Phase Diagram Solutions:
1. Dilute proper amounts of Batch A and Batch B each with half of the total amount of water needed to q.s. the formula to 100%.
2. Mix each until uniform.
3. Pour diluted Batch A solution into diluted Batch B solution.
4. Mix solution until uniform.

The following Table 1 indicates those blends that were formulated and their resulting response (see legend). These responses were then graphed to illustrate regions of compatibility where clear solutions resulted and regions of complexation where it was evident that the two polymers interacted forming a milky-white, colloidal dispersion. This is illustrated in the phase diagram in FIG. 1 entitled “Phase Diagram—Gantrez S-97/Conditioneze NT-20”. This phase diagram located suitable ratios of the two polymers as a complex for split end repair.

TABLE 1
Polymer Complex Mixture Design

| | | | | Prediction |
|---|---|---|---|---|
| NT-20 | S-97 | | Response | Formula |
| (active) | (active) | Water | See legend | Response |
| 0.1 | 0.05 | 99.85 | 5 | 4.387673 |
| 0.25 | 0.05 | 99.7 | 5 | 4.207083 |
| 0.5 | 0.05 | 99.45 | 1 | 3.850187 |
| 0.75 | 0.05 | 99.2 | 2 | 3.423401 |
| 1 | 0.05 | 98.95 | 2 | 2.926725 |
| 0.1 | 0.1 | 99.8 | 5 | 4.55923 |
| 0.25 | 0.1 | 99.65 | 5 | 4.395848 |
| 0.5 | 0.1 | 99.4 | 5 | 4.067633 |
| 0.75 | 0.1 | 99.15 | 1 | 3.669529 |
| 1 | 0.1 | 98.9 | 1 | 3.201534 |
| 0.1 | 0.5 | 99.4 | 5 | 5.29483 |
| 0.25 | 0.5 | 99.25 | 5 | 5.269117 |
| 0.5 | 0.5 | 99 | 5 | 5.170352 |
| 0.75 | 0.5 | 98.75 | 5 | 5.001696 |
| 1 | 0.5 | 98.5 | 5 | 4.76315 |
| 0.1 | 0.75 | 99.15 | 5 | 5.179643 |
| 0.25 | 0.75 | 99 | 5 | 5.239974 |
| 0.5 | 0.75 | 98.75 | 5 | 5.284614 |
| 0.75 | 0.75 | 98.5 | 5 | 5.259364 |
| 1 | 0.75 | 98.25 | 5 | 5.164224 |
| 0.1 | 1 | 98.9 | 5 | 4.622198 |
| 0.25 | 1 | 98.75 | 5 | 4.768572 |
| 0.5 | 1 | 98.5 | 5 | 4.956618 |
| 0.75 | 1 | 98.25 | 5 | 5.074774 |
| 1 | 1 | 98 | 5 | 5.123039 |
| 1 | 0.025 | 98.975 | 5 | 2.782687 |
| 1 | 0.035 | 98.965 | 5 | 2.840833 |
| 1.25 | 0.05 | 98.7 | 3 | 2.360159 |
| 1 | 0.2 | 98.8 | 5 | 3.69808 |
| 1.8 | 0.2 | 98 | 1 | 1.914394 |
| 1 | 0.3 | 98.7 | 5 | 4.123865 |
| 1 | 0.35 | 98.65 | 5 | 4.310222 |
| 1 | 0.4 | 98.6 | 5 | 4.478888 |
| 1 | 0.45 | 98.55 | 5 | 4.629864 |

Response Legend

| Response | Observation |
|---|---|
| 1 | Complex |
| 2 | Very Hazy |
| 3 | Hazy |
| 4 | Slightly Hazy |
| 5 | Clear |

The preferred weight ratio for the two polymers was 0.2% active S-97 and 1.8% active NT-20. These formulas are as follows:

0.2% S-97, 1.8% active NT-20 Complex

| | |
|---|---|
| Appearance: | thin, milky-white liquid |
| pH: | 7.13 |
| Viscosity: | 16.0 (12.5%) (Brookfield DVII + RVT, |
| | UL, 00, 50 rpm, ambient) |
| | 14.5 (22.6%) (Brookfield DVII + RVT, |
| | UL, 00, 100 rpm, ambient) |

The following served as the control formulas:
1% active Gantrez S-97 and 1% active Conditioneze NT-20
2% active Gantrez S-97

2% active Conditioneze NT-20

1% S-97, 1% NT-20 active control Solution

| | |
|---|---|
| Appearance: | slightly hazy serum |
| pH: | 6.88 |
| Viscosity: | 1568(29.4%) (Brookfield DVII + RVT, |
| | LV-2, 60 rpm, ambient) |

Using JMP Statistical Discovery Software Version 3.2, a predictive model was constructed to estimate alternate ratios at which complexation would occur. The prediction equation was based on a mixture design response. As can be seen by the Rsquare value in the following analysis, the prediction equation is poor. However, as is observed in the phase diagram constructed from the prediction equation we learned that the complex forms when there is a low ratio of S-97 to NT-20. This occurs because there are more anionic charges on the Gantrez S-97 molecule than there are cationic charges on the Conditioneze NT-20 molecule. This is illustrated in the phase diagram of FIG. 1. This phase diagram work shows a particular ratio of low anionic to high cationic polymer to form the complex. This complex attaches to the hair through its cationicity to permanently mend split ends since it is complexed with an adhesive anionic polymer.

Statistical Analysis

Summary of Fit

| | |
|---|---|
| Rsquare | 0.416247 |
| RSquare Adj | 0.312006 |
| Root Mean Square Error | 1.1999 |
| Mean of Response | 4.294118 |
| Observations (or Sum Wgts) | 34 |

Effect Test

| | | Sum of | | |
|---|---|---|---|---|
| Source | DF | Squares | F Ratio | Prob > F |
| NT-20 | 1 | 0.463712 | 0.3221 | 0.5749 |
| S-97 | 1 | 3.457521 | 2.4015 | 0.1325 |
| Water | 1 | 36.554260 | 25.3891 | <.0001 |
| S-97*NT-20 | 1 | 7.826488 | 5.4360 | 0.0271 |
| Water*NT-20 | 1 | 0.431711 | 0.2998 | 0.5883 |
| Water*S-97 | 1 | 3.460305 | 2.4034 | 0.1323 |

Effect Test

| Sum of Squares | F Ratio | DF | Prob > F |
|---|---|---|---|
| 7.8264884 | 5.4360 | 1 | 0.0271 |

### Prediction Formula Response

0+−56.991862•NT-20+−350.03147•S-97+0.04316314•Water+S-97•NT-20•6.39168166+Water•NT-20•0.55912058+Water•S-97•3.53807028

TABLE 2
Summary of Results:

| | Complex: | Control: | | |
|---|---|---|---|---|
| | 0.2% S-97 | 1% S-97 and | Control: | |
| | and 1.8% | 1% active NT- | 2% active NT- | Control: |
| | active NT-20 | 20 | 20 | 2% S-97 |
| Percent | 65 | 50 | 50 | 75 |
| Mended | | | | |
| (based on | | | | |
| primary-split | | | | |
| mend before | | | | |
| combing) | | | | |
| Percent | 85 | 70 | 50 | 67 |
| Mended That | | | | |
| Stay Mended | | | | |
| (based on | | | | |
| primary-split | | | | |
| mend after | | | | |
| combing) | | | | |

No split ends on tresses were observed before production of split ends as seen with a hand held lens.

It is preferable to assess split end mend with the use of stereomicroscopy, as compared to visual or hand-held magnifying glass observations.

TABLE 3
S-97/NT-20 Complex:
History of mending for each fiber as observed with
hand held lens and unaided eye
(X = end split, = intact end)

| | | |
|---|---|---|
| | After leave | After leave |
| | on | on |
| | treatment | treatment |
| | before | after |
| Before | combing | combing |

| Fiber # | treatment | Lens | Eye | Lens | Eye |
|---|---|---|---|---|---|
| 1 | X | √ | √ | √ | √ |
| 2 | X | √ | √ | X | X |
| 3 | X | √ | √ | √ | √ |
| 4 | X | √ | √ | √ | √ |
| 5 | X | √ | √ | √ | √ |
| 6 | X | √ | √ | √ | √ |
| 7 | X | √ | √ | √ | √ |
| 8 | X | √ | √ | X | X |
| 9 | X | √ | √ | √ | √ |
| 10 | X | √ | √ | √ | √ |
| 11 | X | √ | √ | X | X |
| 12 | X | √ | √ | √ | √ |
| 13 | X | √ | √ | √ | √ |
| 14 | X | √ | √ | √ | √ |
| 15 | X | √ | √ | √ | √ |
| 16 | X | √ | √ | X | √ |
| 17 | X | √ | √ | √ | √ |
| 18 | X | √ | √ | √ | √ |
| 19 | X | √ | √ | √ | √ |
| 20 | X | √ | √ | √ | √ |

S-97/NT-20 Complex-History of mending for each fiber as
observed with lens-Statistics

| | After leave | After leave |
|---|---|---|
| | on treatment | on treatment |
| | before combing | after combing |
| Total Percent Mended | 100 | 80 |
| Percent mended that | — | 80 |
| stayed mended | | |

TABLE 4
S-97/NT-20 Complex - History of mending primary
through tertiary structure for each fiber as
observed with stereomicroscope 20×
(X = end split, √ = intact end, —= not present,
B = broken off) (P = Primary, S = Secondary,
T = Tertiary)

| | | | | After leave on | | | After leave | | |
|---|---|---|---|---|---|---|---|---|---|
| | | | | treatment before | | | on treatment | | |
| | Before treatment | | | combing | | | after combing | | |
| Fiber # | P | S | T | P | S | T | P | S | T |
| 1 | X | — | — | √ | — | — | √ | — | — |
| 2 | X | — | — | √ | — | — | X | — | — |
| 3 | X | — | — | √ | — | — | √ | — | — |
| 4 | X | — | — | X | — | — | X | — | — |
| 5 | X | — | — | √ | — | — | √ | — | — |
| 6 | X | — | — | √ | — | — | X | — | — |
| 7 | X | X | — | X | √ | — | X | √ | — |
| 8 | X | — | — | X | — | — | X | — | — |
| 9 | X | X | — | X | √ | — | X | X | — |
| 10 | X | — | — | √ | — | — | √ | — | — |
| 11 | X | X | — | X | √ | — | X | X | — |
| 12 | X | — | — | X | — | — | X | — | — |
| 13 | X | — | — | √ | — | — | √ | — | — |
| 14 | X | — | — | √ | — | — | √ | — | — |
| 15 | X | — | — | √ | — | — | √ | — | — |
| 16 | X | — | — | √ | — | — | X | — | — |
| 17 | X | — | — | √ | — | — | X | — | — |
| 18 | X | — | — | X | — | — | X | — | — |
| 19 | X | — | — | √ | — | — | √ | — | — |
| 20 | X | — | — | √ | — | — | √ | — | — |

TABLE 5
History of mending for each fiber as observed with
stereomicroscope 20×-Statistics

| | After leave | | | After leave on | | |
|---|---|---|---|---|---|---|
| | on treatment | | | treatment | | |
| | before combing | | | after combing | | |
| | P | S | T | P | S | T |
| Total Percent | 65 | 100 | — | 55 | 33.3 | — |
| Mended | | | | | | |
| Percent mended | — | — | — | 85 | 50 | — |
| that stayed | | | | | | |
| mended | | | | | | |

TABLE 6
Results from Control Experiments
1% S-97, 1% active NT-20 Control - History of mending for
each fiber as observed with stereomicroscope 20×-Statistics

| | After leave on | | | After leave on | | |
|---|---|---|---|---|---|---|
| | treatment | | | treatment | | |
| | before combing | | | after combing | | |
| | P | S | T | P | S | T |
| Total Percent | 50 | 66.7 | — | 35 | 66.7 | — |
| Mended | | | | | | |
| Percent mended | — | — | — | 70 | 100 | — |
| that stayed | | | | | | |
| mended | | | | | | |

TABLE 7
1% S-97, 1% active NT-20 Control - History of mending for each
fiber as observed with lens-Statistics

| | After leave | After leave |
|---|---|---|
| | on treatment | on treatment |
| | before combing | after combing |
| Total Percent Mended | 85 | 60 |
| Percent mended that | — | 70.5 |
| stayed mended | | |

TABLE 8
2% active NT-20 Control - History of mending for each
fiber as observed with stereomicroscope 20×-Statistics

| | After leave on | | | After leave on | | |
|---|---|---|---|---|---|---|
| | treatment | | | treatment | | |
| | before combing | | | after combing | | |
| | P | S | T | P | S | T |
| Total Percent | 50 | 43 | 100 | 25 | 43 | 100 |
| Mended | | | | | | |
| Percent | — | — | — | 50 | 100 | 100 |
| mended that | | | | | | |
| stayed | | | | | | |
| mended | | | | | | |

TABLE 9
2% active NT-20 Control - History of mending for each fiber as
observed with lens-Statistics

| | After leave | After leave |
|---|---|---|
| | on treatment | on treatment |
| | before combing | after combing |
| Total Percent Mended | 85 | 70 |
| Percent mended that | — | 82 |
| stayed mended | | |

TABLE 10
2% S-97 Control - History of mending for each fiber as observed
with stereomicroscope 20×-Statistics

| | After leave on | | | After leave on | | |
|---|---|---|---|---|---|---|
| | treatment | | | treatment | | |
| | before combing | | | after combing | | |
| | P | S | T | P | S | T |
| Total Percent | 75 | 100 | 100 | 50 | 0 | 0 |
| Mended | | | | | | |
| Percent | — | — | — | 66.7 | 0 | 0 |
| mended that | | | | | | |
| stayed | | | | | | |
| mended | | | | | | |

TABLE 11
2% S-97 Control - History of mending for each fiber as observed
with lens-Statistics

| | After leave | After leave |
|---|---|---|
| | on treatment | on treatment |
| | before combing | after combing |
| Total Percent Mended | 95 | 60 |
| Percent mended that | — | 63 |
| stayed mended | | |

A typical split end mending formulation of the invention is shown below in Table 12.

TABLE 12
Split End Mending Formulation

| INGREDIENTS | % W/W | SUPPLIER |
|---|---|---|
| Water | 49.00 | |
| Xanthan Gum (Rhodicare T) | 0.50 | Rhodia |
| PVM/MA Copolymer (Gantrez S-97) 4.00% | 5.00 | ISP |
| active aq. sol'n. neutralized* | | |
| Polyquaternium-28 (Conditioneze ® NT-20) | 45.00 | ISP |
| 4.00% active aq. sol'n.* | | |
| Propylene Glycol (and) Diazolidinyl Urea | 0.50 | ISP |
| (and) lodopropynyl | | |
| Butylcarbamate (Liquid Germall ® Plus) | | |
| | 100.00% | |

Active ratio of PVM/MA Copolymer to Polyquaternim-28 = 0.20:1.80

Procedure
1. Disperse Rhodicare T into room temperature water with moderate to fast sweep agitation. Mix until uniform.
2. In a premix container add 4.00% active aqueous solution of Conditioneze NT-20 and mix with moderate to fast propeller agitation. Add 4.00% active Gantrez S-97 solution to Conditioneze NT-20 solution with moderate to fast propeller agitation. Mix for ten minutes.
3. Add contents of premix to contents of main container with moderate sweep agitation. Mix until uniform.
4. Dissolve Liquid Germall Plus and mix until uniform.
5. Adjust pH to 7.1±0.1 with 10% citric acid solution.

*Formulas and Procedures for Solutions:

4.00% active Gantrez S-97 aq. Sol'n.

| Ingredient | Wt. % |
|---|---|
| D.I. Water | 86.00 |
| 10.00% aq. Solution NaOH | 10.00 |
| Gantrez S-97 | 4.00 |
| | 100.00 |

Procedure: Add 10.00% solution of NaOH to water with moderate propeller agitation. Sprinkle Gantrez S-97 powder into vortex. Mix until uniform. Adjust pH to 6.95±0.05 with NaOH.

4.00% active Conditioneze NT-20 aq. Sol'n.

| Ingredient | Wt. % |
|---|---|
| D.I. Water | 80.00 |
| Conditioneze NT-20 | 20.00 |
| | 100.00 |

Procedure: Add water to container and mix with moderate sweep agitation. Add Conditioneze NT-20 and mix until uniform.

Physical Properties

| | |
|---|---|
| Appearance | White, semi-opaque, thin, lotion |
| pH | 7.19 |
| Viscosity | 1,675 cps (Brookfield LVT spindle #2 @ 12 rpm, 1 min., |
| | 25° C.) |

Packaging
2 oz. PET bottle; lotion pump
Directions: Massage lotion into damp hair concentrating especially at the ends. Comb product through hair and style.

1. A hair care composition for mending damaged hair fibers comprising a polyelectrolyte complex between anionic and cationic polymers.

2. A hair care composition for mending split end fibers according to claim 1 comprising a polyelectrolyte complex between (a) an anionic polymer of polyvinylmethyl/maleic acid copolymer having the formula: 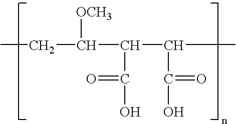
and (b) a cationic polymer of polyquaternium polymer having the formula:

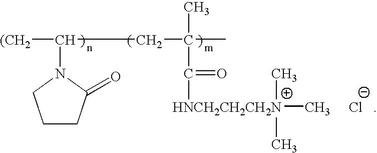

3. A hair care composition according to claim 2 having a low weight ratio of (a) to (b).

4. A hair care composition according to claim 2 wherein the weight ratio of (a):(b) is about 1:1.

5. A hair care composition according to claim 2 wherein the weight ratio of (a):(b) is about 0.2 to 1.8.

6. A hair care composition according to claim 2 wherein said mended split ends remain mended even after combing the hair.

7. A hair care composition according to claim 2 which is a clear to thinly milky-white liquid.

8. A hair care composition according to claim 1 which is a leave-in or rinse-off composition.

9. A hair care composition according to claim 8 which is a shampoo, conditioner, lotion or gel.

10. A process of mending split end fibers in hair tresses which comprises applying thereto the hair care composition of claim 1.

11. A test method for assessment of the degree of split end mending of damaged hair which comprises tagging hair fibers and observing the extent of the repair process by stereomicroscopy.
